(19) United States
(12) Patent Application Publication
(10) Pub. No.: US 2022/0068471 A1
(43) Pub. Date: Mar. 3, 2022

(54) METHOD FOR PROVIDING ERROR INFORMATION IN RESPECT OF A PLURALITY OF INDIVIDUAL MEASUREMENTS

(71) Applicant: Siemens Healthcare GmbH, Erlangen (DE)

(72) Inventor: Robert LAPP, Nuernberg (DE)

(73) Assignee: Siemens Healthcare GmbH, Erlangen (DE)

(21) Appl. No.: 17/411,427

(22) Filed: Aug. 25, 2021

(30) Foreign Application Priority Data
Sep. 1, 2020 (DE) 10 2020 210 995.8

(51) Int. Cl. G16H 40/20; G06N 20/00; G16H 30/20; G16H 70/20; G16H 15/00; G16H 50/70; G16H 50/20

(52) U.S. Cl. CPC G16H 40/20; G06N 20/00; G16H 30/20; G16H 40/40; G16H 15/00; G16H 50/70; G16H 50/20; G16H 70/20

(57) ABSTRACT

A computer-implemented method is for providing error information in respect of a plurality of individual measurements. The method includes providing the plurality of individual measurements, each assigned to one examination respectively; receiving check information from a user in respect of a cohort of examinations to be checked. For each examination of the cohort, the method includes extracting examination information based upon the individual measurements pertaining to the examination; determining, based upon the examination information, whether one or more incorrect measurements has occurred during the examination and ascertaining examination error information based upon the result. The method also includes compiling the error information based upon the examination error information of the cohort to be checked and providing the error information for a user.

### PRIORITY STATEMENT

The present application hereby claims priority under 35 U.S.C. § 119 to German patent application number DE 102020210995.8 filed Sep. 1, 2020, the entire contents of which are hereby incorporated herein by reference.

### FIELD

Example embodiments of the invention generally relate to methods, devices and/or systems for providing error information in respect of a plurality of individual measurements.

### BACKGROUND

Medical examinations of a patient with medical devices are standard methods in medicine. The medical device is frequently an imaging system. Medical examinations of a patient with imaging systems, such as radiography (X-ray imaging) or computed tomography (CT) or magnetic resonance tomography (MRT) or Positron Emission Tomography (PET) or Single Photon Emission Computed Tomography (SPECT) for example, are standard methods in medical diagnostics. At least one medical image of a region of the patient to be examined is acquired or created in the process. In addition, laboratory diagnostic devices are frequently used as medical devices for carrying out a medical examination.

An examination protocol is typically implemented for carrying out a medical examination of this kind with a medical device. The examination protocol comprises one or more individual measurement(s). In other words, the examination protocol provides one or more individual measurement(s) or defines one or more individual measurement(s). The individual measurements of examination protocol differ, for example, in view of measurement settings assigned to them, such as an acquired body region and/or in view of a contrast medium intensification and/or of a measurement instant and/or of a reagent used, etc. The measurement instant can be adjusted, for example, in relation to an instant of a contrast medium dose or in relation to an electrocardiogram signal (ECG) and/or in relation to a movement state of the patient etc.

The reagent can be used, in particular, in a laboratory for examination of a sample from a patient. The sample can be, in particular, a blood sample or a saliva sample or a urine sample or a biopsy sample, etc.

Individual measurements settings can be manually adjusted in the examination protocol by an operator. The operator can be, in particular, a medical technical assistant (MTA) or a medical technical radiology assistant (MTRA) or a specialized medical employee or a chemical technical assistant (CTA) or a physician. In particular, the operator can adjust individual measurement settings to the patient to be examined or the sample to be examined. For example, the operator can set a scan region. The scan region can be set by the operator as a function of the body size of the patient and describes the region, which is to be acquired for the medical examination of the patient with the imaging system. In addition, the operator can manually establish, for example, the measurement instant.

If one of the individual measurements was performed incorrectly, the operator of the medical device can repeat the corresponding individual measurement. An individual measurement that has been carried out incorrectly can also be referred to as an incorrect measurement. The reasons for an incorrect measurement can be, for example, an insufficiently large scan region and/or a scan of an incorrect body region and/or an incorrect measurement instant and/or an insufficient exposure time (for example for observation of a contrast medium bolus) and/or a patient movement and/or an incorrect reagent etc. When repeating the individual measurement, which was performed as an incorrect measurement, in particular the at least one incorrectly chosen measurement setting can therefore be adjusted. In particular, repeating the individual measurement leads to a deviation from the examination protocol, since an additional measurement has to be performed.

In particular, repeating an individual measurement can have adverse effects for the patient. Due to the repetition the patient is thus subjected to additional exposure to radiation in the case of a medical examination based on X-ray radiation. In addition, repeating an individual measurement leads to the length of the examination being extended or repeated sample collection from the patient. This can be unpleasant for the patient. In addition, this reduces the economic efficiency of the medical device. In addition, repeating an individual measurement leads to increased wear of the medical device and/or to increased consumption of laboratory material.

An incorrect measurement usually occurs owing to an error by the operator. An error of this kind can be, for example, incorrect positioning of the patient and/or an incorrectly chosen scan region and/or a different incorrectly chosen measurement setting. Only a small proportion of the incorrect measurements performed in everyday clinical practice is caused by unavoidable events, such as a patient movement, etc.

After the medical examination has finished the individual measurements are typically loaded into a database, for example, into a Picture Archiving and Communication System, (PACS). The operator frequently does not load the incorrect measurements or loads only some of them into the database. It is therefore often difficult to trace the incorrect measurements that have occurred. If incorrect measurements are stored in the database they are also frequently not directly marked as such, and this makes it difficult to trace incorrect measurements that have occurred.

A deviation from the examination protocol can also be desired, however. For example, the operator can use an examination protocol as a template and adjust it to given, patient-specific and/or examination-specific conditions. Such desired deviations should, in particular, not be counted among the incorrect measurements.

### SUMMARY

At least one embodiment of the present invention provides a method, which provides error information in respect of a plurality of individual measurements.

Embodiments are directed to a method for providing error information in respect of a plurality of individual measurements; a method for providing a trained function; a system for providing error information in respect of a plurality of individual measurements; a computer program product; and a computer-readable storage medium. Advantageous developments are specified in the claims and in the following description.

The inventive solutions of the embodiments will be described below both in relation to the devices and in relation to the method. Features, advantages or alternative embodiments mentioned in this connection are likewise to be transferred to the other subject matters, and vice versa. In other words, the concrete claims (which are directed, for example, towards a device) can also be developed with the features, which are described or claimed in connection with a method. The corresponding functional features of the method are developed by corresponding concrete modules.

Furthermore, the inventive solutions of the embodiments are described both in relation to methods and devices for providing error information in respect of a plurality of individual measurements as well as in relation to methods and devices for providing a trained function. Features and alternative embodiments of data structures and/or functions in methods and devices for determination can be transferred to analogous data structures and/or functions in methods and devices for adjusting/optimizing/training. Analogous data structures can be identified here in particular by the use of the prefix “training”. Furthermore, the trained functions used in methods and devices for providing error information in respect of a plurality of individual measurements can have been trained or adjusted and/or provided in particular by methods for providing the trained function.

At least one embodiment of the invention relates to a computer-implemented method for providing error information in respect of a plurality of individual measurements. The method comprises the method step of providing the plurality of individual measurements, which are assigned to one examination respectively. The method also comprises the method step of receiving check information from a user via an interface in respect of a cohort of examinations to be checked. For each examination of the cohort the method steps of extracting examination information based upon the individual measurements pertaining to the examination; determining, based upon the examination information, whether one or more incorrect measurement(s) has/have occurred during the examination; and ascertaining examination error information based upon the result of determining are carried out. The method also comprises the method step of compiling the error information based upon the examination error information of the cohort to be checked. The method also comprises the method step of providing the error information for a user via the interface.

At least one embodiment of the invention also relates to a computer-implemented method for providing a trained function. The method comprises the method step of providing training input data, wherein the training input data comprises at least one item of examination information relating to an examination. The method also comprises the method step of providing training output data, wherein the training output data comprises at least one item of examination error information of the examination. The method also comprises the method step of training the trained function based upon the training input data and the training output data. The method also comprises the method step of providing the trained function.

At least one embodiment of the invention also relates to a system for providing error information in respect of a plurality of individual measurements comprising an arithmetic unit and an interface. The arithmetic unit and/or the interface are designed for providing the plurality of individual measurements, which are assigned to one examination respectively. The interface is also designed for receiving check information from a user in respect of a cohort of examinations to be checked. The arithmetic unit is also designed for extracting examination information for each examination of the cohort based upon the individual measurements pertaining to the examination. The arithmetic unit is also designed for determining for each examination of the cohort, based upon the examination information, whether one or more incorrect measurement(s) has/have occurred during the examination. The arithmetic unit is also designed for ascertaining examination error information for each examination of the cohort based upon the result of determining and the examination information. The arithmetic unit is also designed for compiling the error information based upon the examination error information of the cohort to be examined. The arithmetic unit and/or the interface is/are also designed for providing the error information for a user.

At least one embodiment of the invention also relates to a computer program product with a computer program and to a computer-readable medium. An implementation largely in terms of software has the advantage that even previously used systems for providing error information in respect of a plurality of individual measurements can be easily retrofitted by way of a software update in order to operate in the described manner. A computer program product of this kind can comprise optionally additional elements, such as documentation and/or additional components, as well as hardware components, such as hardware keys (dongles, etc.) in order to use the software, in addition to the computer program.

At least one embodiment of the invention also relates to a computer program product with a computer program, which can be loaded directly into a memory of a system for providing error information in respect of a plurality of individual measurements, with program segments in order to carry out all steps of at least one embodiment of the method for providing error information in respect of a plurality of individual measurements of at least one medical examination and its embodiments if the program segments are run by the system for providing error information in respect of a plurality of individual measurements.

At least one embodiment of the the invention relates to a computer-readable storage medium on which program segments, which can be read and run by a determination system and/or a training system, are stored in order to carry out all steps of at least one embodiment of the method for providing error information in respect of a plurality of individual measurements of at least one medical examination and its embodiments when the program segments are run by the system for providing error information in respect of a plurality of individual measurements.

At least one embodiment of the the invention relates to a computer-implemented method for providing error information in respect of a plurality of individual measurements, comprising:

providing the plurality of individual measurements, each respective individual measurement of the plurality of individual measurements being respectively assigned to one respective examination;

receiving check information from a user via an interface in respect of a cohort of examinations to be checked, for each respective examination of the cohort of examinations to be checked, the method further including:
extracting examination information based upon the respective individual measurement pertaining to the respective examination,
determining, based upon the examination information extracted, whether at least one incorrect measurement has occurred during the respective examination, and
ascertaining examination error information based upon a result of the determining;

compiling the examination error information based upon the examination error information of the cohort of examinations to be checked; and

providing the error information compiled, to a user via the interface.

At least one embodiment of the the invention relates to a computer-implemented method for providing a trained function, comprising:

providing training input data, the training input data including at least one item of examination information on an examination;

providing training output data, the training output data including at least one item of examination error information of the examination and the training output data and the training input data being related;

training the trained function based upon the training input data and the training output data; and

providing the trained function.

At least one embodiment of the the invention relates to a system for providing error information in respect of a plurality of individual measurements, comprising:

an arithmetic unit; and

an interface, at least one of the arithmetic unit and the interface being configured to provide the plurality of individual measurements, each respective individual measurement of the plurality of individual measurements being respectively assigned to one respective examination;

wherein the interface is also configured to receive check information from a user in respect of a cohort of examinations to be checked for each respective examination of the cohort of examinations to be checked,

wherein the arithmetic unit is also configured to
extract examination information based upon the respective individual measurement pertaining to the respective examination,
determine, based upon the examination information extracted, whether at least one incorrect measurement has occurred during the respective examination,
ascertain examination error information based upon a result of the determining, and
compile the examination error information based upon the examination error information of the cohort of examinations to be checked; and

wherein at least one of the arithmetic unit and the interface is also configured to provide the error information for a user.

At least one embodiment of the the invention relates to a non-transitory computer program product storing a computer program, directly loadable into a memory of a system, including program segments to carry out the method of an embodiment upon the program segments being run by the system.

At least one embodiment of the the invention relates to a non-transitory computer-readable storage medium storing program segments, readable and runnable by a system, to carry out the method of an embodiment upon the program segments being run by the system.

### BRIEF DESCRIPTION OF THE DRAWINGS

The above-described properties, features and advantages of this invention will become clearer and more understandable in connection with the following figures and their descriptions of example embodiments. The figures and descriptions are not intended to limit the invention and its embodiments in any way.

Identical components are provided with corresponding reference characters in different figures. As a rule, the figures are not to scale.

In the drawings:

FIG. 1 shows a first example embodiment of a method for providing error information in respect of a plurality of individual measurements,

FIG. 2 shows a second example embodiment of a method for providing error information in respect of a plurality of individual measurements,

FIG. 3 shows a third example embodiment of a method for providing error information in respect of a plurality of individual measurements,

FIG. 4 shows a fourth example embodiment of a method for providing error information in respect of a plurality of individual measurements,

FIG. 5 shows a fifth example embodiment of a method for providing error information in respect of a plurality of individual measurements,

FIG. 6 shows a sixth example embodiment of a method for providing error information in respect of a plurality of individual measurements,

FIG. 7 shows a first example embodiment for providing a trained function,

FIG. 8 shows a second example embodiment for providing a trained function,

FIG. 9 shows a system of an embodiment for providing error information in respect of a plurality of individual measurements,

FIG. 10 shows a training system for providing a trained function.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. At least one embodiment of the present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (procesor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

At least one embodiment of the invention relates to a computer-implemented method for providing error information in respect of a plurality of individual measurements. The method comprises the method step of providing the plurality of individual measurements, which are assigned to one examination respectively. The method also comprises the method step of receiving check information from a user via an interface in respect of a cohort of examinations to be checked. For each examination of the cohort the method steps of extracting examination information based upon the individual measurements pertaining to the examination; determining, based upon the examination information, whether one or more incorrect measurement(s) has/have occurred during the examination; and ascertaining examination error information based upon the result of determining are carried out. The method also comprises the method step of compiling the error information based upon the examination error information of the cohort to be checked. The method also comprises the method step of providing the error information for a user via the interface.

The medical examination is carried out, in particular, on or with a medical device. The medical device can comprise, in particular, an imaging system or a medical imaging system. In particular, at least one medical image of a patient or of a region or of a body region of the patient is created or acquired or measured during the medical examination with the imaging system. The imaging system can have, in particular, an X-ray device, a flat panel X-ray device, a computed tomography device (CT), a magnetic resonance tomography device (MRT), a Position Emission Tomography device (PET), a Single Photon Emission Computed Tomography device (SPECT), a mammography device, etc. and/or a combination of these devices.

Alternatively, the medical device can be a laboratory diagnostic device. In particular, a sample from the patient can be analyzed with the laboratory diagnostic device during the medical examination. In particular, the sample from the patient can be a blood sample or a saliva sample or a urine sample or a biopsy sample, etc. In particular, the sample can have been taken from the patient in advance of the medical examination.

Carrying out the medical examination comprises, in particular, creating or acquiring or recording or measuring one or more individual measurement(s). An individual measurement describes an individual measuring process with the medical device. An individual measurement can be performed, in particular, without interruption. In particular, the measurement settings can be constant or unchanged during an individual measurement. An individual measurement can comprise, in particular, measurement data and/or metadata relating to the measurement data. The metadata is described in more detail below. The measurement data can comprise, in particular, image data and/or data, which was generated or determined during the measuring process. The individual measurements differ, in particular, in relation to their measurement settings. The measurement settings can comprise, or describe or establish or define, for example, an acquired body region of the patient and/or in relation to a scan region and/or a measurement instant and/or an image contrast and/or a sample type and/or a reagent and/or an exposure time and/or an exposure length and/or, in the case of an imaging system based on X-ray radiation, an energy of the X-ray radiation, etc. In particular, the measurement settings can be adjusted to the patient and/or the medical examination. The acquired body region is dependent, in particular, on complaints or on a disease of the patient, for clarification of which the medical examination is being carried out. The scan region, in particular when carrying out tomography as the individual measurement, can be the region of the patient, which is recorded or acquired or represented during the tomography.

The measurement instant can be, in particular, an instant of the recording or performance of the individual measurement as a function of event. The event can be, for example, a contrast medium dose and/or a signal of an electrocardiogram (ECG) and/or a movement state of the patient, etc. In particular, by acquiring a plurality of individual measurements at different measurement instants after a dose of contrast medium it is possible to observe, for example, how a contrast medium bolus spreads. The image contrast can be, in particular, a weighting during an MRT scan. The sample type can describe, in particular, the type of the sample, for example, blood sample, saliva sample, urine sample, biopsy sample, etc. The reagent can be, in particular, a substance, which is used for analysis of the sample.

In particular, an operator can manually set or adjust, for example, the scan region and/or the body region and/or other measurement settings during an individual measurement. In particular, the operator positions the patient in or on or in front of the imaging system. The operator can be, in particular, a medical technical assistant (MTA) and/or a medical technical radiology assistant (MTRA) and/or a chemical technical assistant (CTA) and/or a specialized medical employee and/or a physician, etc.

In particular, information relating to an individual measurement can be incorporated or stored in the metadata relating to the individual measurement and/or the assigned examination. In other words, the metadata can comprise information relating to an individual measurement and/or the assigned examination. The metadata can comprise, for example, the measurement settings and/or a consecutive number for each individual measurement and/or information relating to the medical device used and/or information relating to the operator and/or information relating to the patient, etc. The information relating to the medical device used can comprise, in particular, a manufacturer and/or a year of construction and/or a maintenance condition, etc. in relation to the medical device. The information relating to the operator can comprise, in particular, a name of the operator and/or an encoded name of the operator, etc. The information relating to the patient can comprise, for example, a name of the patient, a weight, a height, a disease, a reason for the examination, etc. The metadata can comprise, in particular, a DICOM header and/or a dose structured report. In particular, the metadata can be queried, for example, via a DICOM Query and Retrieve.

In particular, single individual measurements can be incorrect measurements. In other words, an individual measurement can be an incorrect measurement. An incorrect measurement is an individual measurement that has been carried out incorrectly. In particular, the incorrect measurement can have been carried out or performed with at least one incorrect measurement setting. With an incorrect measurement, for example an incorrect scan region and/or an incorrect body region and/or an incorrect measurement instant and/or an insufficient exposure time and/or an incorrect reagent can have been chosen or set. An incorrect measurement can also occur owing to a movement of the patient during the individual measurement. An incorrect measurement is indicated, in particular, in that the corresponding individual measurement is repeated, is therefore performed twice. The corresponding individual measurement is therefore performed once as the incorrect measurement and subsequently once again with corrected measurement settings as the correct or successful individual measurement.

In particular, the individual measurements can be transferred or loaded from the medical device into a database. In particular, the individual measurements can be archived in the database. In particular, the individual measurements transferred into the database can be referred to as archived individual measurements. The database can be, in particular, a central database. In particular individual measurements of a plurality of medical devices can be stored in the central database. In particular, individual measurements of a plurality of medical examinations can be stored in the central database. The central database can be stored or saved, in particular, on a server or in a Cloud system. The server can be installed, in particular, in a facility, for example a hospital, in which the medical device is located. Alternatively, the server can be an external server. The central database can comprise, in particular, a Picture Archiving and Communication System (PACS). In particular, the individual measurements can be manually transferred by the operator or be automatically transferred into the database. In particular, the operator can choose which individual measurement of the medical examination should be transferred into the database. In particular, the operator can transfer correctly performed or successful individual measurements. In particular, the operator can transfer incorrect measurements into the database only partially or not at all.

In the method step of providing the plurality of individual measurements, in particular those individual measurements can be provided, which are stored or saved in the database. In other words, those individual measurements can be provided, which were transferred into the database. In particular, the plurality of individual measurements can comprise correctly performed or successful individual measurements as well as incorrect measurements. Each of the individual measurements can be assigned to a medical examination. In other words, each examination in the database can comprise one or more individual measurement(s). In particular, one or more examination(s) are stored or saved in the database. In particular, a plurality of medical examinations are stored or saved in the database. In particular, the expression “the database comprises, for example, a plurality of examinations”, hereinafter means that, for example, the plurality of examinations is stored or saved in the database.

In the method step of receiving check information, check information provided or input by a user in respect of a cohort of examinations to be checked is received. The cohort comprises, in particular, one or more medical examination(s). The individual measurements assigned to the examinations of the cohort are provided in particular in the method step of providing a plurality of individual measurements. The user can be, in particular, a person who wants to analyze an occurrence of incorrect measurements. The interface can be, in particular, a user interface. The interface can be, for example, a keyboard and/or a graphical user interface (GUI) and/or a touch screen, etc.

By way of the check information the user can define or determine a cohort of examinations to be checked. In other words, using the check information those examinations can be determined from the plurality of examinations, which are to be assigned to the cohort to be checked. In particular, the cohort to be checked can comprise a subset of the plurality of examinations in the database. In particular, the cohort to be checked can comprise no or all examinations of the plurality of examinations in the database.

In particular, the operator can manually choose the examinations, which should be assigned to the cohort or added to the cohort. Alternatively or in addition, the check information can indicate, for example, that all examinations, which were carried out with a medical device from a particular manufacturer, should be assigned to the cohort. Alternatively or in addition, the check information can indicate, for example, that all examinations in relation to a disease and/or all examinations, which were carried out by a particular operator, etc. should be assigned to the cohort.

In particular, by way of the check information it is possible to filter the plurality of examinations according to any information contained in the individual measurements or in the metadata incorporated by the individual measurements. An examination can be assigned to the cohort or not based upon this filter. In particular, by assigning an examination to the cohort all individual measurements of the examination incorporated by the database can be assigned to the cohort. In alternative embodiments, the check information can be pre-set or be determined automatically.

In the method step of extracting the examination information, the examination information is extracted for each examination of the cohort based upon the individual measurements pertaining to the examination. In particular, the examination information can be extracted from the metadata incorporated by the individual measurements. The examination information can comprise a number of individual measurements in the database, which can be assigned to the examination.

Alternatively or in addition, the examination information can comprise a number for each individual measurement assigned to the examination and incorporated by the database. Alternatively or in addition, the examination information can comprise, for example, a mean pixel value and/or a result or a diagnosis from an individual measurement. In particular, the examination information can comprise any information provided by the metadata relating to the examination or individual measurements assigned to the examination and incorporated by the database.

In the method step of determining, for each examination of the cohort it is determined based upon the examination information whether one or more incorrect measurement(s) has/have occurred during the examination. In other words, it is determined based upon the examination information whether one or more of the individual measurement(s) assigned to the examination and incorporated by the database is an incorrect measurement. In particular, no individual measurement can be an incorrect measurement. In particular, half of the individual measurements assigned to the examination can be incorrect measurements. In this case, each individual measurement was performed once as an incorrect measurement and as a corrected successful individual measurement.

In the method step of ascertaining the examination error information, the examination error information is ascertained for each examination of the cohort based upon the determining. In particular, the examination error information can comprise information about whether at least one incorrect measurement occurred during the examination. In particular, the examination error information can comprise information about whether more than one incorrect measurement occurred during the examination. In particular, the examination error information can comprise information about the number of incorrect measurements during the medical examination. In particular, the examination error information can comprise information about which individual measurement of the examination was performed as an incorrect measurement. In particular, the examination error information can comprise information about which measurement setting was corrected or changed or adjusted during the incorrect measurement compared to the associated correctly performed or successful individual measurement. In other words, the examination error information can comprise information about why the incorrect measurement was faulty.

In the method step of compiling the error information, the error information can be compiled based upon the examination error information of the examinations of the cohort. In other words, error information can be compiled for the entire cohort to be checked. In particular, the error information is based on the examination error information of the examinations, which are incorporated by the cohort to be checked. In particular, examination error information was ascertained for each examination of the cohort to be checked. In particular, the error information can be based on a statistical evaluation and/or a comparison of the examination error information. In particular, the examination information of the examinations can be evaluated for compiling the error information. In other words, the error information can also be based on the examination information. In particular, the error information can indicate, for example, how frequently incorrect measurements occur in the examinations of the cohort. In particular, the error information can comprise information about which individual measurement were performed particularly frequently as an incorrect measurement and/or what the reasons are for an incorrect measurement, etc. In particular, the error information can comprise information about with which operator and/or with which medical device incorrect measurements occur particularly frequently.

In the method step of providing the error information, the user is provided with the error information via the interface. In particular, the user can be provided with the error information via a display unit. In particular, providing can occur via a GUI. In other words, the interface can be designed as a display unit and/or as a GUI. Alternatively, the error information can be saved on a memory, so the user can retrieve the error information at any time. The memory can be, in particular, an internal memory, for example a hard disk and/or a temporary memory, for example a working memory. Alternatively, the memory can be an external memory, for example a database or a Cloud system or a USB memory device or an SD card or a CD or DVD or an external hard disk, etc.

The inventor has found that with the described method the incorrect measurements that have occurred in a cohort can be analyzed. The inventor has also found that it is thus possible to elicit or identify reasons for incorrect measurements. The inventor has found that knowledge about these reasons makes it possible to optimize the carrying out of examinations with respect to economic efficiency and/or the exposure of the patient to radiation and/or patient comfort and/or the length of the examination, etc. The inventor has found that a manual analysis of the examinations of a cohort cannot be carried out owing to the large volumes of data. The inventor has found that with the described method steps it is possible to analyze even large volumes of data in a cohort in a relatively short time and to provide a user with error information. The method makes it possible to analyze large, complex volumes of data for compiling error information. The inventor has found that this is made possible in particular by extracting the examination information.

According to one embodiment of the invention, the error information comprises a statistical evaluation of the examination error information of the cohort in particular in relation to a frequency of incorrect measurements on a medical device and/or to a frequency of incorrect measurements due to an operator and/or a frequency of incorrect measurements in the case of an examination of a particular disease.

In particular, the error information can comprise information about a frequency of incorrect measurements in relation to a manufacturer of the medical device. Alternatively or in addition, the error information can comprise a frequency of incorrect measurements in relation to a type of a medical device. A type can be, in particular, a flat panel X-ray device, a CT device, an MRT device, a PET device, a SPECT device, a particular laboratory diagnostic device, etc. In particular, the error information can comprise information about a frequency of incorrect measurements as a function of a maintenance condition of the one medical device.

Alternatively or in addition, the error information can comprise information about a frequency of incorrect measurements in relation to an operator, etc. In other words, the error information can comprise information about which operator performs incorrect measurements particularly frequently.

Alternatively or in addition, the error information can comprise information about a frequency of incorrect measurements in relation to a particular disease of the patient being examined and/or a plurality of patients being examined. In particular, in this connection the frequency of incorrect measurements in relation to a medical examination typically carried out in the case of this disease can also be incorporated by the error information.

In particular, for the statistical evaluation, the examination information of the examinations of the cohort can be evaluated in addition to the examination error information.

The inventor has found that it is therefore possible to evaluate with which medical device or by which operator incorrect measurements are particularly frequently performed. In particular, the inventor has found that particular operators can have targeted additional training or in the case of a new acquisition, a medical device from a particular manufacturer may be preferred based upon the error information. In addition, the inventor has found that with knowledge from the error information it is possible to purposefully optimize examinations, for example in relation to a particular disease.

According to a further embodiment of the invention, the examination comprises a first and a second set of individual measurements. The first set comprises the successful individual measurements. The second set comprises the incorrect measurements that have occurred. The examination information comprises at least one item of information relating to the individual measurements of the first set and at least one item of information relating to a subset of the individual measurements of the second set.

In particular, the operator and/or the medical device transfers the correctly performed or successful individual measurements of an examination into the database. The successful individual measurements of the examination are, in particular, part of the first set of individual measurements. The incorrect individual measurements that have occurred or incorrect measurements of the examination are transferred, in particular, not at all or only in part or partially or fully transferred manually by the operator or automatically into the database. The incorrect measurements of the examination are in particular part of the second set of individual measurements. The subset of the second set comprises, in particular, only those incorrect measurements of the examination, which were transferred into the database. The subset can comprise, in particular, no incorrect measurement or an incorrect measurement or a plurality of incorrect measurements or all incorrect measurements of the second set. If the subset comprises all incorrect measurements of the second set, the subset comprises, in particular, all incorrect measurements that occurred during the examination. The examination information of the examination therefore comprises at least one item of information about all successful individual measurements. The examination information of the medical examination also comprises at least one item of information about those incorrect measurements of the examination, which are part of the subset of the second set. The information incorporated by the examination information can be formed as described above.

In particular, the archived individual measurements comprise all successful individual measurements of the first set of individual measurements and the incorrect measurements of the subset of the second set of individual measurements. The individual measurements of the first and of the second set of individual measurements as a whole can be referred to as individual measurements that have been carried out. In particular, the individual measurements carried out therefore comprise all individual measurements which were carried out during the examination.

The inventor has found that the examination information comprises at least one item of information relating to the individual measurements of the first set and the subset of the second set. The inventor has found that it must be taken into consideration that the examination information possibly does not comprise at least one item of information relating to each individual measurement of the examination, but only relating to the individual measurements transferred into the database. The inventor has found that this non-uniform transfer of individual measurements into the database makes manual compiling of the error information impossible.

According to a further embodiment of the invention, the first set of individual measurements is specified in an examination protocol. The method also comprises the method step of providing the examination protocol. The step of determining whether one or more incorrect measurement(s) occurred during the examination takes place based upon the examination protocol.

The examination protocol defines the individual measurements, which should be successfully carried out during a medical examination. In particular, the examination protocol can define or establish or specify an order of the individual measurements. The examination protocol can comprise, in particular, the measurement settings for carrying out or performing or acquiring an individual measurement. An incorrect measurement is therefore not incorporated by the examination protocol. In particular, the examination protocol can be pre-defined. In particular, the examination protocol can be adjusted by the operator. In particular, the operator can create the examination protocol. In particular, the operator can adjust the measurement settings via the examination protocol. In particular, the operator can correct the measurement settings via the examination protocol if an incorrect measurement has occurred. In particular, a number of the individual measurements incorporated by the examination protocol does not change as a result of correcting. In particular, during correcting only the measurement settings of the individual measurement to be corrected are adjusted. In particular, the examination protocol can be configured specifically for an examination of a patient for a particular disease or illness. In particular, the examination protocol can be configured specifically for a particular medical device.

In the method step of providing, the examination protocol is provided. In particular, the examination protocol for each examination of the cohort to be checked is provided. In particular, the examination protocol can be provided via the database.

The method step of determining whether one or more incorrect measurement(s) occurred during the examination can then take place based upon the examination protocol.

The inventor has found that the examination protocol comprises information as to how the examination should ideally progress. In other words, the inventor has found that the examination protocol describes how the examination should be carried out without incorrect measurements. In particular, the inventor has found that the examination protocol comprises information about the successful individual measurements.

According to a further embodiment of the invention, the examination information comprises at least one of the following items of information about the individual measurements of the first set and the subset of the second set: designation of the individual measurement, individual measurement type, measured body region in the individual measurement, dose-length product of the individual measurement, name of the examination protocol, reason for the examination.

In particular, the examination information comprises at least one of the items of information listed above for all individual measurements of an examination transferred into the database. In other words, the examination information comprises at least one of the items of information listed above for the first set and the subset of the second set of individual measurements. The designation of the individual measurement can be, in particular, a name of an individual measurement. The designation can have, in particular, descriptive properties. The designation can be, for example, as follows: “skull CT”. In particular, the designation can be unique for the individual measurements of the first set of individual measurements.

The individual measurement type can describe, in particular, how the individual measurement is performed. The type can be, for example, “spiral acquisition” or “acquisition with constant angle” or “T1 weighted scan”, etc. The measured body region indicates which body region is examined or acquired with the individual measurement. The dose-length product indicates, in particular, a radiation dose applied to the patient, In particular, the dose-length product describes the exposure to radiation of a patient as a result of radiography with a CT device. The X-ray voltage of the individual measurement indicates which voltage is applied to an X-ray source emitting the X-ray radiation. In particular, the X-ray voltage therefore comprises information about an X-ray spectrum used for acquisition of a medical image. In particular, the name of the examination protocol can be unique.

In particular, the name of the examination protocol can be descriptive. In other words, the name of the examination protocol can be descriptive. In particular, the name of the examination protocol can describe for the purpose of diagnosis or examination of which disease the examination protocol is being performed. Alternatively or in addition, the name of the examination protocol can comprise a term describing the examination as a whole, for example, “thorax CT”. The reason for the examination can indicate, in particular, why the examination is being carried out. In particular, the reason for the examination can indicate for the purpose of diagnosis or examination of which disease the examination is being carried out. In other words, the reason for the examination can indicate in which context the examination is being carried out. In particular, the disease can be the reason for the examination.

The inventor has found that from the information listed above, which can be incorporated by the examination information of an examination, it is possible to derive or determine whether one or more incorrect measurement(s) has/have occurred.

According to a further embodiment of the invention, the method step of determining comprises whether one or more incorrect measurement(s) has/have occurred comprises the method step of determining a difference between a protocol number of individual measurements and the examination number of individual measurements. The protocol number of individual measurements comprises the number of individual measurements in the first set of individual measurements. The examination number of individual measurements comprises the number of individual measurements in the first set of individual measurements and the subset of the second set of individual measurements. The examination error information is based on the difference in the protocol number and the examination number.

The protocol number of individual measurements comprises, in particular, the number of individual measurements, which are ideally carried out during the examination. In other words, the protocol number comprises the minimum number of individual measurements carried out during the examination if no incorrect measurement occurs. The protocol number of individual measurements comprises, in particular, the number of successful individual measurements in the first set of individual measurements. The protocol number can be determined, in particular, based upon the examination protocol. In other words, the protocol number of individual measurements comprises the number of individual measurements defined in the examination protocol.

The examination number comprises, in particular, the number of successful individual measurements of the first set added to the number of incorrect measurements of the subset of the second set. If the subset is identical to the second set, the examination number comprises the number of individual measurements actually carried out during the examination. In particular, the examination number of individual measurements comprises the number of successful individual measurements plus the number of incorrect measurements. If the subset does not comprise all incorrect measurements of the second set of individual measurements, the examination number of individual measurements comprises the number of successful individual measurements plus the incorrect measurements, which were transferred into the database. In other words, the examination number comprises the number of archived individual measurements.

In the method step of determining the difference, the protocol number of measurements is subtracted from the examination number of measurements. Alternatively, the examination number of measurements can be subtracted from the protocol number of measurements. In particular, the number of incorrect measurements in the subset of the second set of individual measurements can thus be determined. In particular, the difference can describe the total number of incorrect measurements during the examination if the subset comprises the total second set of individual measurements.

In particular, the examination error information can be based on the difference. In particular, the examination error information can comprise information about whether the difference is equal to or different from zero. In particular, the examination error information can comprise the difference. In particular, the examination information can comprise information about a number of provided incorrect measurements in relation to a medical examination. In particular, the examination error information can comprise information about a number of incorrect measurements of the examination transferred into the database. If all incorrect measurements were transferred into the database or if all incorrect measurements were provided or if the subset is the same as the second set, the examination error information can therefore comprise information about a total number of incorrect measurements that have occurred during the examination.

The inventor has found that, in particular if all individual measurements are transferred into the database, a total number of incorrect measurements during an examination can thus be determined. In addition, the inventor has found that it is then also possible to determine whether at least one incorrect measurement has even occurred. The inventor has found that this information can be incorporated by the examination error information. In addition, the inventor has found that this information can be determined for each examination of the cohort to be checked.

According to a further embodiment of the invention, a consecutive number is assigned to each individual measurement of the first and second sets. The examination information comprises the numbers of the individual measurements of the first set and the subset of the second set. The method step of determining whether one or more incorrect measurement(s) has/have occurred comprises the method step of checking whether the numbers of the individual measurements in the examination information are consecutive. The examination error information comprises the result of the check.

In particular, a consecutive number can be assigned during an examination of each individual measurement. In particular, a unique number is therefore assigned to each individual measurement of the first set and each individual measurement of the second set. In particular, the number for each examination can be unique. In addition, the number for a medical device can be unique. The numbers can describe, in particular, the order of the individual measurements. The examination information can comprise, in particular, the consecutive numbers of those individual measurements, which were provided. In particular, the examination information can comprise the consecutive numbers of those individual measurements, which were transferred into the database. In other words, the examination information can comprise, in particular, the consecutive numbers of the individual examinations of the of the quantity and the subset of the second set.

In the method step of checking, it is checked whether the numbers in the examination information are consecutive. If the numbers in the examination information are not consecutive, it is possible to infer that there is at least one incorrect measurement, which was not provided. In particular, it is then possible to infer that there is at least one incorrect measurement, which was not transferred into the database. In particular, it is possible to infer that there is at least one incorrect measurement in the second set, which is not part of the subset. In particular, when checking it is possible to determine how many “gaps” there are in the consecutive series of consecutive numbers and thus infer the number of incorrect measurements that have not been provided or transferred. In particular, in the method step of checking, it is possible to infer the total number of incorrect measurements during the medical examination if no incorrect measurement was provided or transferred into the database. In other words, it is possible to infer the total number of incorrect measurements if the subset does not comprise any incorrect measurement of the second set.

The result of the method step of checking can comprise, in particular, information about whether there is at least one incorrect measurement. Alternatively or in addition, the result can comprise information about the number of incorrect measurements that have not been provided or transferred into the database. The examination error information can comprise, in particular, the result of checking.

The inventor has found that based upon the consecutive number of the individual measurements it is possible to identify or check whether incorrect measurements have occurred if the incorrect measurements have not been provided or been transferred into the database. The inventors also found that this method is of interest especially if no incorrect measurements or only a few incorrect measurements are transferred into the database. In particular, the inventor has found that by a combination of the above-described method steps for determining the examination error information, the total number of incorrect measurements of an examination can be determined irrespective of whether the incorrect measurements were transferred into the database or not. In other words, the total number of incorrect measurements can be determined by way of a combination of the method steps of determining the difference between the protocol number of individual measurements and the examination number of individual measurements and checking whether the numbers of the individual measurements in the examination information are consecutive irrespective of whether all, none or only some of the incorrect measurements have been transferred into the database.

According to a further embodiment of the invention, an incorrect measurement of the second set of individual measurements corresponds to an individual measurement of the first set of individual measurements. The examination error information comprises information, which indicates which of the individual measurements of the first set has a corresponding incorrect measurement in the subset of the second set.

In particular, as described above, each incorrect measurement is an incorrect individual measurement or an incorrectly performed individual measurement. In particular, those individual measurements can be established in the examination protocol, which are to be successfully carried out for carrying out the examination. Since the individual measurement should be successfully or correctly carried out for a complete examination, the incorrect individual measurement is carried out again or repeated following correction or adjusting of the measurement parameters or the settings. In particular, the individual measurement is then successfully carried out. In particular, the successful individual measurement is part of the first set of individual measurements. In particular, the successful individual measurement then corresponds to the incorrect measurement. In other words, the incorrect measurement corresponds to the successful individual measurement. In particular, with an incorrect measurement and the corresponding successful individual measurement, the majority of the measurement settings can be identical. In particular, only manually adjustable measurement settings of the incorrect measurement and the corresponding successful individual measurement can differ from each other. In particular, the reason or an aim for which the individual measurement should be carried out remains unchanged or the same in the case of the incorrect measurement and the corresponding successful individual measurement. In particular, the designation of the incorrect measurement and the corresponding successful individual measurement is the same or unchanged.

In particular in respect of each individual measurement of the first set and the subset of the second set, the examination information comprises at least one item of information, which is the same or unchanged or identical in the case of an incorrect measurement and the corresponding successful individual measurement. In particular, this at least one item of information can be uniquely assigned to the incorrect measurement and the corresponding successful individual measurement. In particular, the at least one item of information of the individual measurement is incorporated in the examination protocol. In particular in respect of each individual measurement of the first set and the subset of the second set, the examination information can comprise a combination of information, which in the case of an incorrect measurement and the corresponding successful individual measurement are the same or unchanged or identical. In particular, the combination of information can be uniquely assigned to the incorrect measurement and the corresponding successful individual measurement. In particular based upon the at least one item of information or the combination of information, a successful individual measurement from the first set can then be assigned to an incorrect measurement of the subset of the second set. In particular, the mutually corresponding incorrect measurements and successful individual measurements can thus be assigned to each other. In particular if the subset corresponds to the second set, a successful individual measurement from the first set can be assigned to each incorrect measurement that has occurred.

The inventor has found that it is thus possible to determine which individual measurement from the examination protocol is particularly error-prone. In other words, in the described manner it is possible to determine which individual measurement specified in the examination protocol is performed as an incorrect measurement. From this it is possible to infer, in particular, which individual measurement specified in the examination protocol is particularly frequently incorrect, is therefore carried out as an incorrect measurement. In other words, it is possible to derive which individual measurement is repeated particularly frequently.

According to a further embodiment of the invention, a designation is assigned to each individual measurement of the first and second sets. The designations of the individual measurements of the first set are unique. The designation of an incorrect measurement of the second set corresponds to the designation of the corresponding individual measurement of the first set. The examination information comprises the designations of the individual measurements of the first set of individual measurements and the subset of the second set of individual measurements. The method step of determining whether one or more incorrect measurement(s) has/have occurred comprises the method step of determining identical designations in the examination information. The examination error information comprises information based on identical designations.

In particular, for each examination of the cohort, the examination error information comprising the information based upon the identical designations is ascertained in the examination information of the corresponding examination.

In particular, the designation of the corresponding individual measurement remains unchanged in comparison to the designation of the corresponding incorrect measurement when carrying out the successful individual measurement with a corrected measurement setting or corrected measurement parameters. In particular, the designation can be defined or specified in the examination protocol for an individual measurement. In particular, the individual measurement specified in the examination protocol can have been performed as a successful individual measurement and/or as an incorrect measurement. In particular, the designation can be a name of the individual measurement. In particular, the designation can comprise a reason for or an aim of the individual measurement. In particular, the designation within the first set of individual measurements can be uniquely assigned to an individual measurement. In particular, the designation within the second set of individual measurements can be uniquely assigned to an incorrect measurement. In particular, the corresponding successful individual measurement of the first set can be assigned to an incorrect measurement based upon the designation. In particular, the examination information comprises the designations of the successful individual measurements from the first set and the designations of the incorrect measurements from the subset of the second set.

In the method step of determining identical designations in the examination information, matching designations in the examination information are determined. In other words, in the method step, designations of individual measurements are determined, which occur multiple times in the examination information. In particular, designations are determined, which occur twice. Since a designation of an individual measurement specified in the examination protocol can be uniquely assigned, the designation can also be uniquely assigned to an individual measurement in the first set. In particular, it follows from this that in the case of a duplicate or identical designation in the examination information, the corresponding individual measurement is included both in the first set of individual measurements and in the subset of the second set of individual measurement. In other words, a designation can only occur multiple times in the examination information if the individual measurement corresponding to the designation occurs both in the first set and in the subset of the second set.

The examination error information can be based, in particular, on the same designations. In particular, the examination error information can indicate which of the individual measurements of the first set were initially carried out incorrectly as an incorrect measurement. In particular, the examination error information can comprise identical designations. Alternatively or in addition, the examination error information can comprise a number of identical designations in the examination information.

The inventor has found that known information, such as the designation of an individual measurements, can be used to provide information about which individual measurement was carried out incorrectly. In particular, the inventor has found that it is thus possible to determine which individual measurement was particularly frequently carried out incorrectly or as an incorrect measurement. In other words, it is thus possible to determine which individual measurement was repeated.

According to a further embodiment of the invention, the method step of determining whether one or more incorrect measurement(s) has/have occurred comprises the method step of applying a trained function to the examination information, whereby the examination error information is generated.

In particular, the examination information comprises the measurement settings of the individual measurements from the first set and the subset of the second set.

In the method step of applying the trained function, the examination error information is generated via the trained function based upon the examination information. In particular, the examination error information is ascertained for each examination of the cohort via the trained function.

In general, a trained function imitates cognitive functions, which people associate with human thought. In particular, by way of training based on training data, the trained function can adjust to new circumstances and identify and extrapolate patterns.

In general, parameters of a trained function can be adjusted via training. In particular, supervised training, semi-supervised training, unsupervised training, reinforcement learning and/or active learning can be used for this. Furthermore, representation learning (an alternative term is feature learning) can be used. In particular, the parameters of the trained functions can be iteratively adjusted by a plurality of training steps.

In particular, a trained function can comprise a neural network, a support vector machine, decision tree and/or a Bayesian network, and/or the trained function can be based on k-means clustering, Q-Learning, genetic algorithms and/or association rules. In particular, a trained function can comprise a combination of a plurality of uncorrelated decision trees or an ensemble of decision trees (random forest). In particular, the trained function can be determined via XGBoosting (eXtreme Gradient Boosting). In particular, a neural network can be a deep neural network, a convolutional neural network or a convolutional deep neural network. Furthermore, a neural network can be an adversarial network, a deep adversarial network and/or a generative adversarial network. In particular, a neural network can be a recurrent neural network. In particular, a recurrent neural network can be a network with long-short-term-memory (LSTM), in particular a Gated Recurrent Unit (GRU). In particular, a trained function can comprise a combination of the described approaches. In particular, the approaches described here are cited for a trained function network architecture of the trained function.

The inventor has found that the examination error information can be determined via a trained function based upon the examination information.

According to a further embodiment of the invention, the trained function comprises a first trained sub-function and a second trained sub-function. The first trained sub-function comprises an unsupervised learning algorithm. The second trained sub-function comprises a classification algorithm. A result of the first trained sub-function is input into the second trained sub-function.

In particular, the examination information of an examination of the cohort is used as input data for the first trained sub-function. In particular, the output data of the first trained sub-function is used as input data for the second trained sub-function. In particular, the second trained sub-function outputs the examination error information of the examination as output data.

In particular, the examination information can comprise the above-described information. In particular, the examination information comprises the above-described information for the successful individual measurements from the first set and the incorrect measurements from the subset of the second set. In particular, categoric data in the examination information can be converted via One-Hot Encoding into binary data. In particular, categoric data can be information in the examination information, which can assume only two states, for example, on/off; yes/no, etc. For example, information about a contrast medium dose can be categoric information. In particular, this information can assume only the expression “yes” (contrast medium was administered) and “no” (no contrast medium was administered).

In particular, the first trained sub-function can be designed to identify outlier. In particular, the first trained sub-function can be designed to recognize outliers in the examination information. In particular, an outlier can be an unusual value of a measurement setting in the examination information. In particular, the value of an outlier can deviate significantly from corresponding values from other examinations. In particular, an outlier can be, for example, an unusually large scan region in the case of an individual measurement. In particular, an outlier of a measurement setting of an individual measurement can point to the fact that the corresponding individual measurement is an incorrect measurement. In particular, the first trained sub-function can be based on a k-means algorithm (k-means clustering) and/or on a Gaussian mixture model or Gaussian mixture algorithm.

In particular, the second trained sub-function can comprise a classification algorithm. In particular, the second trained sub-function can be based on an ensemble of decision trees (random forest) and/or a support vector machine. The second trained sub-function is designed, in particular, to filter outliers, which were identified with the first trained sub-function, but which were intentionally or knowingly generated or measured by the operator. In particular, outliers intentionally generated by the operator are not an indication of an incorrect measurement. In particular, the operator can consciously choose, for example, a large scan region in order to have greater certainty during the examination. In particular, this is then not an incorrect measurement.

The inventor has found that the combination of the first trained sub-function and the second trained sub-function ensures that the examination error information does not comprise any outliers or only a few incorrectly identified as an incorrect measurement. The inventor has found that annotated second training output data is necessary only for training the second trained sub-function. The inventor has also found that by applying an unsupervised learning algorithm to training of the first trained sub-function, no annotated first training output data is necessary. The inventor has found that it is possible to generate annotated second training output data more easily and time-efficiently based upon the output data of the first trained sub-function than based upon the examination information. In other words, the inventor has found that training of the entire trained function can be optimized or accelerated or simplified by the combination of the first and second sub-functions.

At least one embodiment of the invention also relates to a computer-implemented method for providing a trained function. The method comprises the method step of providing training input data, wherein the training input data comprises at least one item of examination information relating to an examination. The method also comprises the method step of providing training output data, wherein the training output data comprises at least one item of examination error information of the examination. The method also comprises the method step of training the trained function based upon the training input data and the training output data. The method also comprises the method step of providing the trained function.

In particular, the training output data can be manually created. In particular, the training input data can be annotated or marked or identified by an expert for this. In other words, incorrect measurements can be identified in the training input data by the experts for determining the training output data. Alternatively, successful individual measurements can be identified in the training input data in order to determine or generate the training output data. In particular, the training output data can be automatically determined or generated.

In particular, training can take place in two stages. In particular, training takes place in two stages if the trained function comprises a first trained sub-function and a second trained sub-function as described above.

In particular, the first trained sub-function can be trained in a first step or a first stage. In particular, the first trained sub-function is trained based upon training input data. Sub-training output data is generated or determined in the process. The first sub-training output data comprises, in particular, information on outliers in measurement settings of an individual measurement incorporated by the examination information. In particular, the first sub-training output data can be automatically determined or ascertained by way of unsupervised learning. Alternatively, the first sub-training output data can be manually annotated.

In particular, the second trained sub-function can then be trained in a second step or in a second stage. In particular, the second trained sub-function is trained based upon the first sub-training output data as the second sub-training input data and the training output data. In particular, the training output data can be determined by an expert from the second sub-training input data or the first sub-training output data by way of manual annotation. In particular, the second trained sub-function can be trained by way of supervised training.

The inventor has found that two-stage training is an efficient method for training a trained function for automatic determining of the examination error information of an examination.

According to one embodiment of the invention, the trained function is continuously further trained via feedback. The feedback is provided by a user of the examination error information.

In particular, the user of the examination error information is also the user of the error information. In particular, the user can indicate via user interface in the form of user information whether he is satisfied with the provided result in respect of the error information. In particular, the user can provide corrected error information about the user interface as user information. In particular, the user information can serve as training output data for training the trained function.

The inventor has found that the trained function can thus be continuously trained further and optimized in an application.

At least one embodiment of the invention also relates to a system for providing error information in respect of a plurality of individual measurements comprising an arithmetic unit and an interface. The arithmetic unit and/or the interface are designed for providing the plurality of individual measurements, which are assigned to one examination respectively. The interface is also designed for receiving check information from a user in respect of a cohort of examinations to be checked. The arithmetic unit is also designed for extracting examination information for each examination of the cohort based upon the individual measurements pertaining to the examination. The arithmetic unit is also designed for determining for each examination of the cohort, based upon the examination information, whether one or more incorrect measurement(s) has/have occurred during the examination. The arithmetic unit is also designed for ascertaining examination error information for each examination of the cohort based upon the result of determining and the examination information. The arithmetic unit is also designed for compiling the error information based upon the examination error information of the cohort to be examined. The arithmetic unit and/or the interface is/are also designed for providing the error information for a user.

A system of this kind for providing error information in respect of a plurality of individual measurements can be designed, in particular, to carry out the above-described method for providing error information in respect of a plurality of individual measurements of at least one medical examination and its embodiments. The system for providing error information in respect of a plurality of individual measurements is designed to carry out this method and its embodiments in that the interface and the arithmetic unit are designed to carry out the corresponding method steps.

At least one embodiment of the invention also relates to a computer program product with a computer program and to a computer-readable medium. An implementation largely in terms of software has the advantage that even previously used systems for providing error information in respect of a plurality of individual measurements can be easily retrofitted by way of a software update in order to operate in the described manner. A computer program product of this kind can comprise optionally additional elements, such as documentation and/or additional components, as well as hardware components, such as hardware keys (dongles, etc.) in order to use the software, in addition to the computer program.

At least one embodiment of the invention also relates to a computer program product with a computer program, which can be loaded directly into a memory of a system for providing error information in respect of a plurality of individual measurements, with program segments in order to carry out all steps of at least one embodiment of the method for providing error information in respect of a plurality of individual measurements of at least one medical examination and its embodiments if the program segments are run by the system for providing error information in respect of a plurality of individual measurements.

At least one embodiment of the the invention relates to a computer-readable storage medium on which program segments, which can be read and run by a determination system and/or a training system, are stored in order to carry out all steps of at least one embodiment of the method for providing error information in respect of a plurality of individual measurements of at least one medical examination and its embodiments when the program segments are run by the system for providing error information in respect of a plurality of individual measurements.

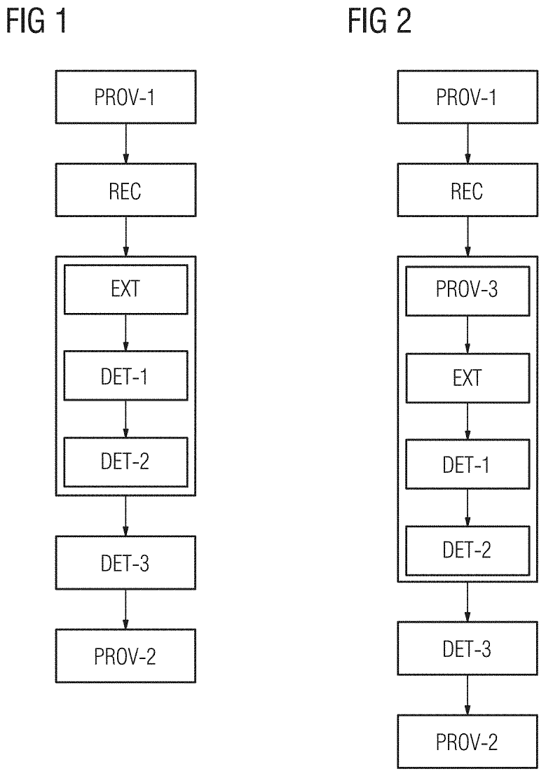

FIG. 1 shows a first example embodiment of a method for providing error information in respect of a plurality of individual measurements.

In the method step of providing PROV-01 of a plurality of individual measurements, the plurality of individual measurements is provided, for example, by a database. The individual measurements can be assigned to one examination respectively. In other words, an examination comprises one or more individual measurement(s). The examination can be performed or carried out with a medical device. The individual measurements assigned to the examination are acquired or recorded or measured with the medical device. In particular, the individual measurements can be transferred automatically or manually into the database. Alternatively, providing PROV-01 can occur directly by way of the medical device. The medical device can be, in particular, an imaging system such as a flat panel-X-ray device or a CT device or an MRT device or a PET device or a SPECT device or a mammography X-ray device, etc. Alternatively, the medical device can be a laboratory diagnostic device. In particular, the plurality of individual measurements can have been acquired with more than one medical device.

An examination is performed in particular on a patient. An examination comprises one or more individual measurement(s). The individual measurements of an examination can differ, for example, in respect of a measurement setting such as an acquired body region of the patient and/or a scan region and/or a measurement instant and/or a reagent used and/or an exposure time and/or an exposure length and/or an image contrast and/or in the case of an imaging system based on X-ray radiation, an energy of the X-ray radiation, etc. In particular, the measurement settings can be adjusted to the patient and/or the medical examination. An individual measurement can be a successful individual measurement or an incorrect measurement. An incorrect measurement is an incorrect individual measurement. In particular, with an incorrect measurement, one or more measurement setting(s) can be incorrectly set or chosen. In particular, an incorrect individual measurement or an incorrect measurement with corrected measurement settings is repeated. In particular, the plurality of individual measurements comprises a plurality of successful individual measurements and optionally one or more incorrect measurement(s). In particular, the plurality of individual measurements for each incorrect measurement can then comprise a corresponding successful individual measurement. The corresponding successful individual measurement is the repeated individual measurement of the incorrect measurement with corrected measurement settings. In particular, an examination comprises a first set of individual measurements and a second set of individual measurements. In particular, the first set of individual measurements comprises all successful or correctly carried out measurements, which are assigned to the examination. In particular, the second set of individual measurements comprises the incorrect measurements, which are assigned to the examination. In particular, an individual measurement of the first set can be assigned to each incorrect measurement of the second set. In particular, the second set can be empty. Alternatively, the second set can comprise as many individual measurements as the first set.

An individual measurement can comprise, in particular, measurement data and/or metadata relating to the individual measurement.

In particular, the measurement data can be image data. In particular, the measurement data can be data, which was acquired or measured or determined during the individual measurement.

The metadata comprises information in respect of the examination or the individual measurements assigned to the examination. In particular, the metadata can be assigned by way of the individual measurement incorporating metadata to one examination respectively. In particular, the metadata can comprise, for example, a DICOM header and/or a Dose Structured Report. In particular, the metadata can be queried, for example, via a DICOM Query and Retrieve. Alternatively, the metadata can be, for example, a laboratory protocol, etc. In particular, the metadata can comprise information about one or more measurement setting(s) and/ or about a medical device, which was used for the examination, and/or about an operator, who carried out the examination, and/or about a patient, on which the examination was carried out, etc.

The database can be, in particular, an internal database. Alternatively, the database can be an external database. In particular, the database can be a central database. The central database can be stored or saved, for example, on a server and/or in a Cloud system. For image data the central database can comprise, for example, a Picture Archiving and Communication System (PACS). Individual measurements from a plurality of medical devices can be loaded or transferred into the central database. In particular, all successful individual measurements incorporated by the first set of individual measurements are transferred into the database. In particular, a subset of the second set of individual measurements is transferred into the database. In other words, a subset of the incorrect measurements of an examination that have occurred are transferred into the database. In particular, the subset can be empty. Alternatively, the subset can correspond to the second set. In particular, the operator can determine which incorrect measurement should be transferred into the database. In particular, the subset then comprises those incorrect measurements, which are transferred into the database. In particular, all or no incorrect measurements can be automatically transferred into the database.

In the method step of receiving REC check information, check information is received from a user receives via an interface in respect of a cohort of examinations. The individual measurements, which are assigned to the examinations incorporated by the cohort, are provided, in particular, in the method step of providing PROV-1 a plurality of individual measurements. In particular, the user can be a person, who analyzes an occurrence of incorrect measurements. The interface can be designed, in particular, as a keyboard and/or GUI and/or touch screen. Via the check information the user can establish criteria or check criteria according to which examinations should be assigned to the cohort. For example, criteria can be the manufacturer of the medical device, which was used for carrying out the examination, an operator during the examination, an indication or a reason for an examination etc. In particular, the check information can comprise one or more of these criteria.

In the method step of extracting EXT examination information, the examination information is extracted based upon the individual measurements pertaining to the examination. In particular, the examination information can be extracted from the metadata incorporated by the individual measurements. In particular, the examination information comprises about the individual measurements incorporated by the examination. Alternatively or in addition, the examination information comprises information about the examination. In particular, the examination information can comprise information about the manufacturer of the medical device with which the examination was carried out. In particular, the examination information can comprise information about the operator, who carried out the examination. In particular, the examination information can comprise at least one of the following items of information: designation of the individual measurement, type of the individual measurement, examination number of the individual measurements, number of the individual measurement, measured body region during the individual measurement, dose-length product of the individual measurement, X-ray voltage of the individual measurement, name of the examination protocol, reason for the examination.

In the method step of determining DET-1, it is determined based upon the examination information for each examination of the cohort whether one or more incorrect measurement(s) has/have occurred. In particular, for the examination, for which the method step is being carried out, it is checked whether one or more incorrect measurement(s) has/have occurred during the examination. In other words, in this method step it is determined whether one or more individual measurement(s) of the examination was/were performed incorrectly.

In the method step of ascertaining DET-2 examination error information, the examination error information for each examination of the cohort is ascertained based upon the result of determining DET-1. In particular, the examination error information of an examination comprises information as to whether at least one incorrect measurement occurred during the examination. In particular, the examination error information of an examination can comprise information about how many incorrect measurements occurred during the course of the examination. In particular, the examination error information comprises information about which of the individual measurements of the examination was performed incorrectly.

The method steps of extracting EXT the examination information, of determining DET-1 whether one or more incorrect measurement(s) occurred during an examination and ascertaining DET-2 the examination error information are performed or carried out in particular for each examination of the cohort of examinations. In order to clarify this a large box is depicted around the corresponding method steps in the illustration in FIG. 1, which box includes the corresponding method steps.

In the method step of compiling DET-3 error information, the error information is compiled based upon the examination error information of the cohort to be checked. In particular, the examination error information of the cohort for compiling DET-3 the error information can be compared and/or statistically evaluated. In particular, the error information can also be based on the examination information of the cohort. In particular, the error information can indicate, for example, which individual measurements were carried out incorrect particularly frequently and/or with which medical device incorrect measurements occur particularly frequently and/or with which operator incorrect measurements occur particularly frequently and/or with which indication or reason for an examination or in relation to which disease or illness incorrect measurements occur particularly frequently and/or what the reasons for the incorrect measurements were. In relation to the medical device the error information can comprise, in particular, information about an occurrence of incorrect measurements as a function of a manufacturer of the medical device and/or a maintenance condition of the medical device and/or a year of construction of the medical device and/or a series of medical devices and/or a type of medical devices, etc. The reasons for the incorrect measurements can be determined, in particular, by way of a comparison of the incorrect measurement and of the corresponding successful individual measurement. In particular, a changed measurement setting in the case of the successful individual measurement compared to the incorrect measurement can point to this measurement setting having been the reason for the incorrect measurement.

In the method step of providing PROV-2 the error information, the error information is provided for a user via an interface. In particular, the user can be provided with the error information via a display unit. The display unit can be, particular, a screen or a computer screen. In particular, the error information can be displayed on the display unit in a GUI. Alternatively, the error information can be saved on a storage medium from which the user can retrieve the error information. The storage medium can be, in particular, a hard disk or an SD card or a CD or a DVD or a USB stick, etc. In particular, the error information can be stored in a database from which the user can retrieve the error information. Alternatively, the error information can be sent to the user via e-mail (electronic mail) and/or SMS (Short Message Service), etc.

FIG. 2 shows a second example embodiment of a method for providing error information in respect of a plurality of individual measurements.

The method steps of providing PROV-1 a plurality of individual measurements, of receiving REC check information, of extracting EXT examination information, of determining DET-1 whether one or more incorrect measurement(s) has/have occurred, of ascertaining DET-2 examination error information, of compiling DET-3 error information and of providing PROV-2 the error information are performed analogously to the description relating to FIG. 1.

In the method step of providing PROV-3 an examination protocol, an examination protocol is provided for each examination of the cohort. In particular the successful individual measurements incorporated by the examination are defined or specified in the examination protocol. In other words, the first set of individual measurements of an examination is specified or defined in the examination protocol. In particular, the corresponding measurement settings are defined in the examination protocol for each individual measurement of the examination. In particular, an examination protocol can be configured for an examination of a particular disease. In particular, the examination protocol defines an ideal progression of the examination. In particular, the examination protocol defines a number of individual measurements which are necessary as a minimum in order to carry out the examination. In particular, the operator can adjust one or more measurement setting(s) in the examination protocol. In particular, the operator can correct the corresponding measurement setting in the examination protocol in order to correct an incorrect measurement. In particular, the number of specified individual measurements does not change in the examination protocol.

In particular, the method step of determining DET-1 whether one or more incorrect measurement(s) occurred during the examination can also be based on the examination protocol.

In alternative embodiments, a plurality of examination protocols can be provided in the method step of providing PROV-1 the plurality of individual measurements. In particular, an examination can then be assigned to each examination protocol. In other words, an examination protocol can be assigned to each examination.

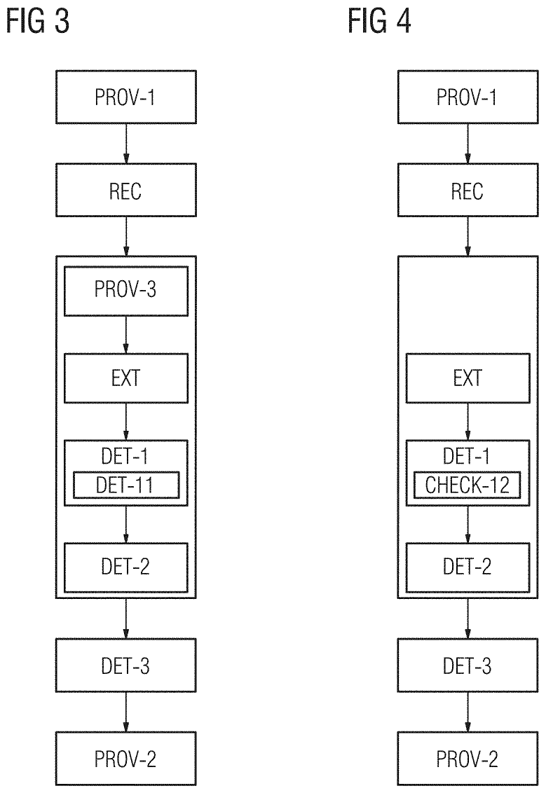

FIG. 3 shows a third example embodiment of a method for providing error information in respect of a plurality of individual measurements.

The method steps of providing PROV-1 a plurality of individual measurements, of receiving REC check information, of extracting EXT examination information, of determining DET-1 whether one or more incorrect measurement(s) has/have occurred, of ascertaining DET-2 examination error information, of compiling DET-3 error information and of providing PROV-2 the error information are performed analogously to the description relating to FIG. 1. The method step of providing PROV-3 an examination protocol takes place according to the description relating to FIG. 2.

The method step of determining DET-1 whether one or more incorrect measurements has/have occurred comprises the method step of determining DET-11 a difference. In the method step of determining DET-11 the difference, the difference between a protocol number of individual measurements and an examination number of individual measurements is determined. In particular, the protocol number of individual measurements corresponds to the number of individual measurements, which are specified in the examination protocol. The examination number of individual measurements of an examination corresponds to the number of individual measurements in the first set of individual measurements added to the number of individual measurements in the subset of the second set of individual measurements. In particular, the examination number of individual measurements corresponds to the number of individual measurements, which were provided in relation to the examination in the method step of providing PROV-1 a plurality of individual measurements. In particular, the examination number corresponds to the number of individual measurements of the examination, which were transferred into the database. In particular, the difference corresponds to the number of incorrect measurements in the subset of the second set of individual measurements. In particular, the difference corresponds to a total number of incorrect measurements of the examination if the subset of the second set is the same as the second set. In other words, the difference corresponds to the total number of incorrect measurements if all incorrect measurements were transferred into the database. In particular, the difference corresponds to the total number of incorrect measurements if all incorrect measurements of the examination were provided in the step of providing PROV-1 a plurality of individual measurements.

The examination error information can then be based, in particular, on the difference. In particular, the examination error information can include the difference.

FIG. 4 shows a fourth example embodiment of a method for providing error information in respect of a plurality of individual measurements.

The method steps of providing PROV-1 a plurality of individual measurements, of receiving REC check information, of extracting EXT examination information, of determining DET-1 whether one or more incorrect measurement(s) has/have occurred, of ascertaining DET-2 examination error information, of compiling DET-3 error information and of providing PROV-2 the error information are performed analogously to the description relating to FIG. 1.

The method step of determining DET-1 whether one or more incorrect measurement(s) has/have occurred comprises the method step of checking CHECK-12. In the method step of checking CHECK-12, it is checked whether numbers of the individual measurements in the examination information are consecutive. In particular, a consecutive number is assigned to each individual measurement of the examination. In other words, each individual measurement of the first and of the second set comprises a unique number. In particular, the examination information comprises the numbers of the individual measurements, which were provided in the step of providing PROV-1 the plurality of individual measurements in relation to the examination. In other words, the examination information comprises the numbers of the individual measurements of the first set and the subset of the second set. In particular, incorrect measurements that have not been provided or transferred can be identified or determined if their number is not included in the examination information. In particular, incorrect measurements that have not been provided are identified or determined by “gaps” in the numbers of the examination information. In particular, all incorrect measurements can be identified if the subset of the second set is empty. In particular, a number of incorrect measurements can thus be determined based upon the missing numbers.

In particular, the examination error information can then be based on this number of incorrect measurements. In particular, the examination error information can comprise the number of incorrect measurements.

In particular, this example embodiment can be combined with the third example embodiment. In particular, the total number of all incorrect measurements that have occurred during an examination can then be determined irrespective of whether they are part of the subset of the second set of individual measurements or not.

FIG. 5 shows a fifth example embodiment of a method for providing error information in respect of a plurality of individual measurements.

The method steps of providing PROV-1 a plurality of individual measurements, of receiving REC check information, of extracting EXT examination information, of determining DET-1 whether one or more incorrect measurement(s) has/have occurred, of ascertaining DET-2 examination error information, of compiling DET-3 error information and of providing PROV-2 the error information are performed analogously to the description relating to FIG. 1.

The method step of determining DET-1 whether one or more incorrect measurement(s) has/have occurred comprises the method step of determining DET-13 identical designations. In the method step of determining DET-13 identical designations, identical designations for individual measurements in the examination information are determined. In other words, designations in the examination information are determined, which occur two or more times. In particular, the examination information comprises a designation for each individual measurement provided in the method step of providing PROV-1 a plurality of individual measurements. The designation is unique for the corresponding individual examination. The designations or mutually corresponding individual measurements are identical. In particular, the designation of an incorrect measurement is identical to the corresponding successful individual measurement. In particular, the corresponding individual measurement is a repetition of the incorrect measurement with corrected measurement settings. In particular, the examination information comprises the designations of the individual measurements of the first set and the individual measurements of the subset of the second set. In particular, each individual measurement is ideally performed only once during an examination with a unique designation. In particular, from two identical designations in the examination information it can be inferred that one of the two designations designates an incorrect measurement. In particular, if can also be inferred which individual measurement was performed once as an incorrect measurement.

The examination error information comprises, in particular, information, which is based on the identical designations. In particular, the examination information can comprise information about how many identical designations could be determined in the examination information. In particular, the examination error information can then comprise information about the number of incorrect measurements in the subset of the second set. Alternatively or in addition, the examination error information can comprise information about which individual measurements were performed as incorrect measurements. In particular, the examination error information can comprise identical designations occurring two or more times.

In particular, this example embodiment can be combined with the third and/or fourth example embodiment(s).

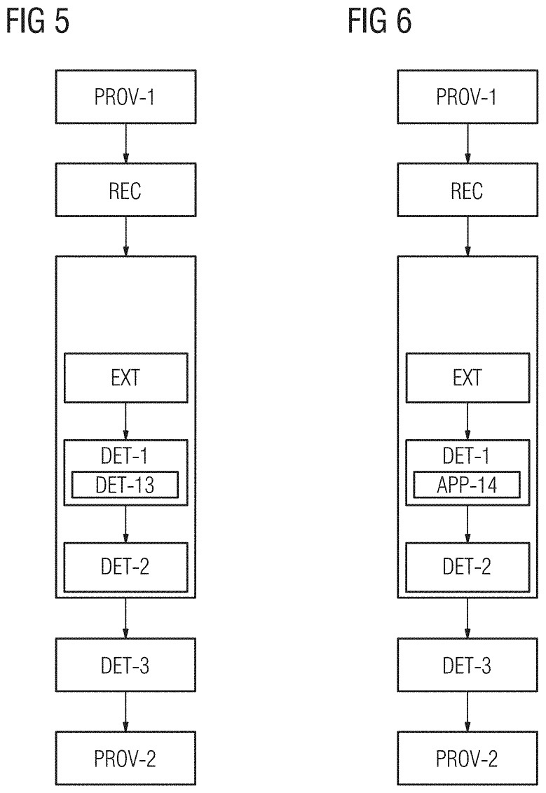

FIG. 6 shows a sixth example embodiment of a method for providing error information in respect of a plurality of individual measurements.

The method steps of providing PROV-1 a plurality of individual measurements, of receiving REC check information, of extracting EXT examination information, of determining DET-1 whether one or more incorrect measurement(s) has/have occurred, of ascertaining DET-2 examination error information, of compiling DET-3 error information and of providing PROV-2 the error information are performed analogously to the description relating to FIG. 1.

The method step of determining DET-1 whether one or more incorrect measurement(s) has/have occurred comprises the method step of applying APP-14 a trained function to the examination information. The examination error information is generated in the process. In particular, the input data of the trained function comprises the examination information. In particular, the output data of the trained information comprises the examination error information. In particular, the trained function can comprise a first trained sub-function and a second trained sub-function. In particular, the first trained sub-function can comprise an unsupervised or non-supervised learning algorithm. In particular, the first trained sub-function can be based on a k-means algorithm (k-means clustering) and/or on a Gaussian mixture model or Gaussian mixture algorithm. In particular, the second trained sub-function can comprise a classification algorithm. In particular, the second trained sub-function can be based on an ensemble of decision trees (random forest) and/or a support vector machine. In particular, the output data of the first trained sub-function can serve as input data of the second trained sub-function. In particular, outliers can be detected in the examination information with the first trained sub-function. In other words, individual measurements can be identified in the examination information with the first trained sub-function, which at least one measurement setting, which significantly deviate from measurement settings used as standard. In particular, a deviation of this kind can point to an incorrect measurement. In particular, a deviation of this kind can sometimes be intended by an operator and does not point to an incorrect measurement. In particular, the second trained sub-function can be designed to identify whether it is a desired or intentional deviation or is an incorrect measurement.

In particular, the examination error information comprises the result of the trained function. In particular, the examination error information can comprise information about the number of incorrect measurements and/or about which individual measurement was performed as an incorrect measurement and/or which measurement setting led to the incorrect measurement, etc. In particular, it is possible to infer from the deviation which measurement setting led to the incorrect measurement.

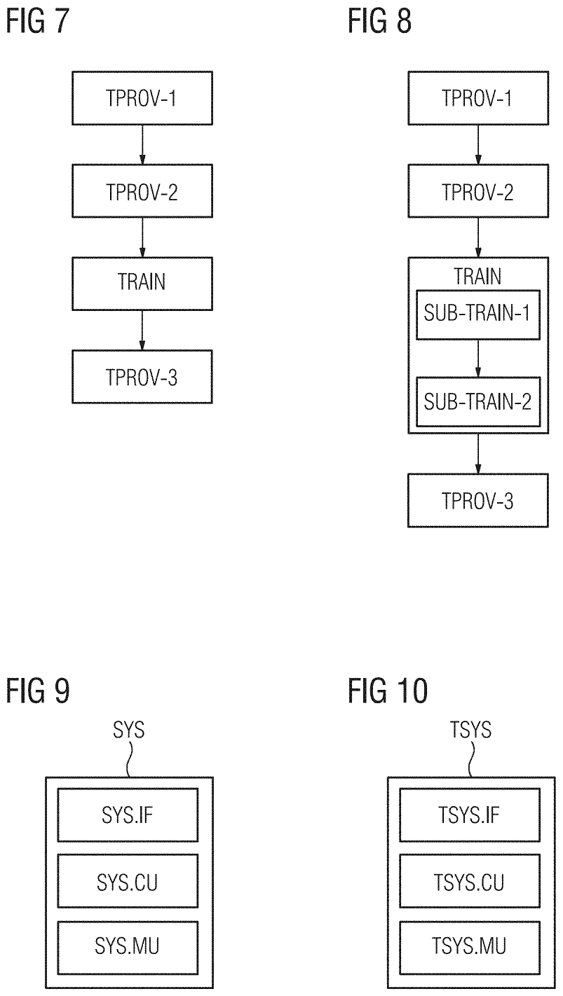

FIG. 7 shows a first example embodiment for providing a trained function.

In the method step of providing TPROV-1 training input data, the training input data for training the trained function is provided. The training input data comprises at least one item of examination information relating to an examination. The examination information can be formed as described above.

In the method step of providing TPROV-2 training output data, the training output data is provided for training the trained function. The training output data comprises at least one item of examination error information of the examination.

In particular, the training output data can be based on the training input data. In other words, the training output data can be related to the training input data. In particular, the training output data can be determined manually and/or automatically from the training input data. In particular, in the case of manual determining, an expert can determine the training output data based upon the training input data. In particular, the expert can determine the training output data by annotating the training input data.

In the method step of training TRAIN the trained function, the trained function is trained based upon the training input data and the training output data. In particular, output data is determined from the training input data via the trained function. In other words, output data is determined or generated or ascertained by applying the trained function to the training input data. In particular, this output data is compared with the training output data. In particular, the trained function is adjusted in such a way that the output data as far as possible matches the training output data. This is referred to, in particular, as “supervised learning”.

In the method step of providing TPROV-2 the trained function, the trained function is provided for application to unknown input data with unknown output data.

FIG. 8 shows a second example embodiment for providing a trained function.

The method steps of providing TPROV-1 the training input data, of providing TPROV-2 the training output data and of providing TPROV-3 the trained function are performed analogously to the description in FIG. 8.

The method step of training TRAIN the trained function comprises the method step training SUB-TRAIN-1 the first trained sub-function and training SUB-TRAIN-2 the second trained sub-function.

The first and the second trained sub-function can be configured as described in the description relating to FIG. 7. The first trained sub-function is trained in the method step of training SUB-TRAIN-1 the first trained sub-function in particular via unsupervised learning. The second trained sub-function is trained in the method step of training SUB-TRAIN-2 the second trained function in particular via supervised learning. In particular, the training input data according to the description relating to FIG. 7 serves as training input data of the first trained sub-function. In particular, the sub-training output data of the first trained function serves as sub-training input data of the second trained function. In particular, the training output data according to the description relating to FIG. 7 serves as training output data of the second trained sub-function. In particular, the training output data can be determined manually and/or automatically from the sub-training input data of the second trained function. In particular, supervised learning can occur analogously to the description relating to FIG. 7.

FIG. 9 shows a system SYS for providing error information in respect of a plurality of individual measurements, FIG. 10 shows a training system TSYS for providing trained function.

The illustrated system SYS for providing error information in respect of a plurality of individual measurements is designed to carry out an inventive method for providing error information in respect of a plurality of individual measurements. The illustrated training system TSYS is designed to carry out an inventive method for providing the trained function. The system SYS comprises an interface SYS.IF, an arithmetic unit SYS.CU and a memory unit SYS.MU. The training system TSYS comprises a training interface TSYS.IF, a training arithmetic unit TSYS.CU and a training memory unit TSYS.MU.

The system SYS and/or the training system TSYS can be, in particular, a computer, a microcontroller or an integrated circuit(IC). Alternatively, the system SYS and/or the training system TSYS can be a real or virtual computer network (a technical designation for a real computer network is “Cluster”; a technical designation for a virtual computer network is “Cloud”). The system SYS and/or the training system TSYS can be designed as a virtual system, which is implemented on a computer or a real computer network or a virtual computer-network (a technical designation is “virtualization”).

The interface SYS.IF and/or the training interface TSYS.IF can be a hardware or software interface (for example, a PCI bus, USB or Firewire). The arithmetic unit SYS.CU and/or the training arithmetic unit TSYS.CU can comprise hardware and/or software elements, for example, a microprocessor or what is known as an FPGA (Field Programmable Gate Array). The memory unit SYS.MU and/or the training memory unit TSYS.MU can be designed as a non-permanent working memory (Random Access Memory, RAM) or as a permanent mass memory (hard disk, USB stick, SD card, Solid State Disk (SSD)).

The interface SYS.IF and/or the training interface TSYS.IF can comprise, in particular, a plurality of sub-interfaces, which perform different method steps of the respective inventive method. In other words, the interface SYS.IF and/or the training interface TSYS.IF can be designed as a plurality of interfaces SYS.IF and/or training interfaces TSYS.IF. The arithmetic unit SYS.CU and/or the training arithmetic unit TSYS.CU can comprise, in particular, a plurality of sub- arithmetic units, which perform different method steps of the respective inventive method. In other words, the arithmetic unit SYS.CU and/or the training arithmetic unit TSYS.CU can be designed as a plurality of arithmetic units SYS.CU and/or training arithmetic units TSYS.CU.

Where it has not explicitly occurred but is expedient and within the meaning of the invention, individual example embodiments, individual partial aspects or features thereof can be combined with each other or interchanged without departing from the scope of the present invention. Advantages of the invention described with reference to one example embodiment also apply without being explicit mentioned, and where transferable, to other example embodiments.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. § 112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A computer-implemented method for providing error information in respect of a plurality of individual measurements, comprising:
providing the plurality of individual measurements, each respective individual measurement of the plurality of individual measurements being respectively assigned to one respective examination;
receiving check information from a user via an interface in respect of a cohort of examinations to be checked, for each respective examination of the cohort of examinations to be checked, the method further including:
extracting examination information based upon the respective individual measurement pertaining to the respective examination,
determining, based upon the examination information extracted, whether at least one incorrect measurement has occurred during the respective examination, and
ascertaining examination error information based upon a result of the determining;
compiling the examination error information based upon the examination error information of the cohort of examinations to be checked; and
providing the examination error information compiled, to a user via the interface.

2. The method of claim 1, wherein the examination error information comprises a statistical evaluation of the examination error information of the cohort of examinations to be checked.

3. The method of claim 1,
wherein the examination comprises a first and a second set of individual measurements,
wherein the first set comprises the successful individual measurements,
wherein the second set comprises the incorrect measurements that have occurred,
wherein the examination information comprises at least one item of information on the individual measurements of the first set, and
wherein the examination information comprises at least one item of information on a subset of the individual measurements of the second set.

4. The method of claim 3,
wherein the first set of individual measurements is specified in an examination protocol,
wherein the method further comprises:
providing the examination protocol, and wherein the determining of whether at least one incorrect measurement has occurred during the examination takes place based upon the examination protocol.

5. The method of claim 3, wherein the examination information comprises at least one item of information about the individual measurements of the first set and the subset of the second set including at least one of:
designation of the individual measurement,
type of the individual measurement,
body region measured during the individual measurement,
dose-length product of the individual measurement,
X-ray voltage of the individual measurement,
name of the examination protocol, and
reason for the examination.

6. The method of claim 5, wherein the determining of whether at least one incorrect measurement has occurred comprises:
determining a difference between a protocol number of individual measurements and the examination number of individual measurements,
wherein the protocol number of individual measurements comprises the number of individual measurements in the first set of individual measurements,
wherein the examination number of individual measurements comprises the number of individual measurements in the first set of individual measurements and the subset of the second set of individual measurements, and
wherein the examination error information is based on the difference in the protocol number and the examination number.

7. The method of claim 3,
wherein a consecutive number is assigned to each respective individual measurement of the first and second sets,
wherein the examination information comprises the numbers of the individual measurements of the first set and the subset of the second set,
wherein the determining of whether at least one incorrect measurement has occurred comprises:
checking whether the numbers of the individual measurements in the examination information are consecutive, wherein the examination error information comprises the result of the checking.

8. The method of claim 3,
wherein an incorrect measurement of the second set of individual measurements corresponds to an individual measurement of the first set of individual measurements,
wherein the examination error information comprises information, indicating which of the individual measurements of the first set has a corresponding incorrect measurement in the subset of the second set.

9. The method of claim 3,
wherein a designation is assigned to each respective individual measurement of the first and second sets,
wherein the designations of the respective individual measurements of the first set are unique,
wherein the designation of incorrect measurement of the second set corresponds to the designation of a corresponding respective individual measurement of the first set,
wherein the examination information comprises the designations of the respective individual measurements of the first set of individual measurements and the subset of the second set of individual measurements,
wherein the determining of whether at least one incorrect measurement has occurred comprises:
determining identical designations in the examination information, wherein the examination error information includes information based upon the identical designations.

10. The method of claim 1, wherein the determining of whether at least one incorrect measurement has occurred comprises:
applying a trained function to the examination information, to generate the examination error information.

11. The method of claim 10,
wherein the trained function includes a first trained sub-function and a second trained sub-function,
wherein the first trained sub-function includes an unsupervised learning algorithm,
wherein the second trained sub-function includes a classification algorithm, and
wherein a result of the first trained sub-function is input into the second trained sub-function.

12. A computer-implemented method for providing a trained function, comprising:
providing training input data, the training input data including at least one item of examination information on an examination;
providing training output data, the training output data including at least one item of examination error information of the examination and the training output data and the training input data being related;
training the trained function based upon the training input data and the training output data; and
providing the trained function.

13. The method of claim 12,
wherein the trained function is continuously trained further via feedback, and
wherein the feedback is provided by a user of the examination error information.

14. A system for providing error information in respect of a plurality of individual measurements, comprising:
an arithmetic unit; and
an interface, at least one of the arithmetic unit and the interface being configured to provide the plurality of individual measurements, each respective individual measurement of the plurality of individual measurements being respectively assigned to one respective examination;
wherein the interface is also configured to receive check information from a user in respect of a cohort of examinations to be checked for each respective examination of the cohort of examinations to be checked,
wherein the arithmetic unit is also configured to
extract examination information based upon the respective individual measurement pertaining to the respective examination,
determine, based upon the examination information extracted, whether at least one incorrect measurement has occurred during the respective examination,
ascertain examination error information based upon a result of the determining, and
compile the examination error information based upon the examination error information of the cohort of examinations to be checked; and
wherein at least one of the arithmetic unit and the interface is also configured to provide the error information for a user.

15. A non-transitory computer program product storing a computer program, directly loadable into a memory of a system, including program segments to carry out the method of claim 1 upon the program segments being run by the system.

16. A non-transitory computer-readable storage medium storing program segments, readable and runnable by a system, to carry out the method of claim 1 upon the program segments being run by the system.

17. The method of claim 2, wherein the examination error information comprises a statistical evaluation of the examination error information of the cohort of examinations to be checked in relation to at least one of a frequency of incorrect measurements on a medical device, a frequency of incorrect measurements due to an operator and a frequency of incorrect measurements during an examination of a particular disease.

18. The method of claim 2,
wherein the examination comprises a first and a second set of individual measurements,
wherein the first set comprises the successful individual measurements,
wherein the second set comprises the incorrect measurements that have occurred,
wherein the examination information comprises at least one item of information on the individual measurements of the first set, and
wherein the examination information comprises at least one item of information on a subset of the individual measurements of the second set.

19. The method of claim 18,
wherein the first set of individual measurements is specified in an examination protocol,
wherein the method further comprises:
providing the examination protocol, and wherein the determining of whether at least one incorrect measurement has occurred during the examination takes place based upon the examination protocol.

20. The method of claim 4, wherein the examination information comprises at least one item of information about the individual measurements of the first set and the subset of the second set including at least one of:
designation of the individual measurement,
type of the individual measurement,
body region measured during the individual measurement,
dose-length product of the individual measurement,
X-ray voltage of the individual measurement,
name of the examination protocol, and
reason for the examination.
